(19) United States
(12) Patent Application Publication
(10) Pub. No.: US 2026/0245666 A1
(43) Pub. Date: Aug. 20, 2026

(54) METHOD AND DEVICE FOR TRAINING MODEL FOR ESTIMATING POLYGENIC RISK SCORE

(71) Applicant: GENOPLAN INC., New York, NY (US)

(72) Inventors: Byung Kyu Kang, Camden, DE (US); Byung Chul Lee, Seoul (KR); Sun Bin Kim, Seoul (KR); Joon Ho Kang, Seoul (KR); Myeong Jae Cheon, Seoul (KR)

(21) Appl. No.: 19/161,142

(22) PCT Filed: Apr. 15, 2024
(86) PCT No.: PCT/KR2024/095732

§ 371 Date: Aug. 29, 2025

(30) Foreign Application Priority Data
May 4, 2023 (KR) 10-2023-0058571
May 23, 2023 (KR) 10-2023-0066517

(51) Int. Cl. G16B 40/20; G06N 3/09; G16B 20/50

(52) U.S. Cl. CPC G16B 40/20; G06N 3/09; G16B 20/50

(57) ABSTRACT

A method of training a model for estimating a polygenic risk score (PRS), according to an embodiment of the present disclosure, may construct a training data set including one or more pieces of training data in which genetic information about a single nucleotide polymorphism (SNP) is an input and a PRS calculation value corresponding to the genetic information is a label. Furthermore, the method may train the model for estimating the PRS, based on the training data set.

### TECHNICAL FIELD

The present disclosure relates to a method and device for training a model for estimating a polygenic risk score.

### BACKGROUND ART

A polygenic risk score (PRS) calculating method is a method of measuring the risk of a specific disease due to inherited factors. Recent studies have been conducted into a method of calculating a PRS by using a deep learning model so that the PRS is calculated by reflecting interaction between single nucleotide polymorphisms (SNPs) included in an SNP list.

Accordingly, to improve the accuracy of a model for calculating a PRS, there is a need to train a deep learning model to assign a weight to each SNP by taking into account the association between SNPs.

The aforementioned background technology is technical information possessed by the inventor for derivation of the present disclosure or acquired by the inventor during the derivation of the present disclosure, and is not necessarily prior art disclosed to the public before the application of the present disclosure.

### DISCLOSURE OF INVENTION

### Technical Problem

Some embodiments according to the present disclosure are directed to providing a method and device for training a model for estimating a polygenic risk score. The problems to be solved by the present disclosure are not limited to those described above, and other problems and advantages of the present disclosure that are not described herein will be understood from the following description and will be more clearly understood from embodiments of the present disclosure. Furthermore, it will be appreciated that the problems to be solved by the present disclosure and the advantages of the present disclosure may be realized by the means indicated in the claims and combinations thereof.

### Solution to Problem

As a technical means for achieving the technical problems described above, a first aspect of the present disclosure may provide a method of training a model for estimating a polygenic risk score (PRS), the method including: constructing a training data set including one or more pieces of training data in which genetic information about a single nucleotide polymorphism(SNP) is an input and a PRS calculation value corresponding to the genetic information is a label; and training the model for estimating the PRS, based on the training data set, wherein the training includes: calculating the genetic information by using the model; obtaining, from the model, a PRS estimation value corresponding to the genetic information; and updating parameters for the model by performing backpropagation on the model, based on a loss function regarding an error between the PRS estimation value and the PRS calculation value, which is the label for the genetic information.

A second aspect of the present disclosure may provide a device for training a model for estimating a polygenic risk score (PRS), the device including: a memory in which at least one program is stored; and a processor configured to execute the at least one program, wherein the processor is further configured to construct a training data set including one or more pieces of training data in which genetic information about a single nucleotide polymorphism(SNP) is an input and a PRS calculation value corresponding to the genetic information is a label, and train the model for estimating the PRS, based on the training data set, wherein the processor is further configured to calculate the genetic information by using the model, obtain, from the model, a PRS estimation value corresponding to the genetic information, and update parameters for the model by performing backpropagation on the model, based on a loss function regarding an error between the PRS estimation value and the PRS calculation value, which is the label for the genetic information.

A third aspect of the present disclosure may provide a computer-readable recording medium having recorded thereon a program for causing a computer to perform the method according to the first aspect.

In addition, other methods and other systems for implementing the present disclosure and computer-readable recording media storing a computer program for performing the methods may be further provided.

Other aspects, features, and advantages of the present disclosure will become better understood through the accompanying drawings, the appended claims, and the detailed description.

### Advantageous Effects of Invention

According to an embodiment of the present disclosure, a model for estimating a polygenic risk score may be prevented from overfitting to training data during a learning process.

More specifically, according to an embodiment of the present disclosure, a model for estimating a polygenic risk score may be trained to assign a weight to each single nucleotide polymorphism (SNP) by taking into account the association between SNPs.

### BRIEF DESCRIPTION OF DRAWINGS

FIG. 1 is a diagram for describing an example of a model for estimating a polygenic risk score (PRS), according to an embodiment.

FIG. 2 is a diagram for describing an example of a training data set used to train a model, according to an embodiment.

FIG. 3 is a diagram for describing an example of a method of training a model based on a training data set, according to an embodiment.

FIG. 4 is a diagram for describing another example of a method of training a model based on a training data set, according to an embodiment.

FIG. 5 is a flowchart for describing an example of a method of training a model for estimating a PRS, according to an embodiment.

FIG. 6 is a configuration diagram for describing an example of an internal configuration of a device for training a model for estimating a PRS, according to an embodiment.

### BEST MODE FOR CARRYING OUT THE INVENTION

A method of training a model for estimating a polygenic risk score (PRS), according to an embodiment of the present disclosure, may include constructing a training data set including one or more pieces of training data in which genetic information about a single nucleotide polymorphism(SNP) is an input and a PRS calculation value corresponding to the genetic information is a label, and training the model for estimating the PRS, based on the training data set. Furthermore, the training may include calculating the genetic information by using the model, obtaining, from the model, a PRS estimation value corresponding to the genetic information, and updating parameters for the model by performing backpropagation on the model, based on a loss function regarding an error between the PRS estimation value and the PRS calculation value, which is the label for the genetic information.

### MODE FOR THE INVENTION

The advantages and features of the present disclosure, and methods of achieving them will be clarified with reference to embodiments described below in detail with reference to the accompanying drawings. However, the present disclosure is not limited to the embodiments presented below and may be implemented in various different forms. Rather, it will be understood that the present disclosure includes all modifications, equivalents, and substitutes falling within the concept and technical scope of the present disclosure. The embodiments presented below are provided so that the present disclosure will be thorough and complete and will fully convey the concept of the present disclosure to those of ordinary skill in the art. In describing the present disclosure, when the detailed description of the relevant known technology is determined to obscure the gist of the present disclosure, the detailed description thereof may be omitted.

The terms as used herein are only used to describe particular embodiments and are not intended to limit the present disclosure. The singular forms as used herein are intended to include the plural forms as well unless the context clearly indicates otherwise. The terms “comprise,” “include,” or “have” as used in the present application are inclusive and therefore specify the presence of one or more stated features, integers, steps, operations, elements, components, or any combination thereof, but do not preclude the presence or addition of one or more other features, integers, steps, operations, elements, components, or any combination thereof.

Some embodiments of the present disclosure may be represented by functional block configurations and various processes. Some or all of such functional blocks may be implemented in any number of hardware and/or software configurations that perform specific functions. For example, the functional blocks of the present disclosure may be implemented by one or more microprocessors or may be implemented by circuit configurations for certain functions. In addition, for example, the functional blocks of the present disclosure may be implemented in various programming or scripting languages. The functional blocks may be implemented as algorithms to be executed by one or more processors. In addition, the present disclosure may employ conventional technologies for electronic environment setting, signal processing, and/or data processing. The terms such as “mechanism,” “element,” “means,” and “configuration” may be used broadly and are not limited to mechanical and physical configurations.

In addition, connecting lines or connecting members illustrated in the drawings are intended to represent functional connections and/or physical or circuit connections. In an actual device, connecting lines or connecting members illustrated in the drawings may represent connections between components by means of a variety of functional, physical, or circuit connections that may be substituted or added.

The term “gene” as used in the present specification refers to a segment of a nucleic acid sequence (also referred to as a “coding sequence” or a “coding region” in the present application) which codes a protein or a ribonucleic acid (RNA), and in some cases, this may be accompanied by a regulatory region, for example, a promoter, an operator, a terminator, or the like, which may be located upstream or downstream of the coding sequence.

The term “genetic information” as used in the present specification encompasses information obtained through genetic analysis of a subject. The genetic information includes, for example, information about genetic traits or genetic mutations associated with the occurrence of a specific disease. The genetic mutations may be in the form of a missense mutation, a frameshift mutation, a nonsense mutation, a splice mutation, a nucleotide substitution, insertion or deletion, or the like, but the present disclosure is not limited thereto. In specific examples, the genetic information may include a single nucleotide polymorphism (SNP). A disease development risk calculated based on the genetic information includes the meaning of an innate development risk for the disease.

In the present specification, a “polymorphism” refers to a case where two or more alleles exist at a single genetic locus. Among the polymorphic sites, a polymorphism in which only a single base differs from person to person is referred to as an SNP. A desirable polymorphic marker has two or more alleles that occur in a selected group at a frequency of 1% or more, and more preferably 5% or 10% or more.

In the present specification, a “polygenic risk score (PRS)” is a genetic evaluation index which evaluates the likelihood that an individual or entity will develop a specific disease by synthesizing the influence of multiple genetic mutations found in the genome of each individual or entity. In general, the PRS may be calculated as a combination of SNPs. In other words, the SNP is a replacement of one base with another base in a deoxyribonucleic acid (DNA) sequence. In case that the location where the replacement of the base occurs is within a gene, a trait or disease associated with the gene may be expressed. Accordingly, the PRS may be calculated as a combination of SNPs associated with a specific disease.

In the present specification, a “model” may refer to a deep learning model based on an artificial neural network (ANN). The deep learning model may have a deep structure with a plurality of hidden layers in the ANN and may learn complex patterns therethrough. For example, the deep learning model may include an input layer, a hidden layer, and an output layer, each of which includes a plurality of neurons. Furthermore, each of the neurons may calculate an output value by applying a value obtained by multiplying an input value by a weight to an activation function, each of the layers may transmit a signal to a next layer, and the signal may be adjusted through the weight and bias. That is, the deep learning model may optimize the weight and the bias by using training data and may learn a complex relationship between an input and an output therethrough. On the other hand, the deep learning model may be implemented as various types of models, such as multi-layer perceptron (MLP), convolutional neural network (CNN), or recurrent neural network (RNN). In addition, because the deep learning model may be implemented as various models according to a method of configuring an activation function, a loss function, an optimization algorithm, or the like, the deep learning model may not be limited to a specific type of model.

In the present specification, “training data” and “training data set” refer to data for training an artificial intelligence model. Each piece of the data may include an input and a label. The “input” refers to data input to the model among pieces of data included in the training data, and the “label” refers to a ground truth for input data and a ground truth to be predicted by the model among pieces of data included in the training data.

Hereinafter, embodiments are described in detail with reference to the accompanying drawings. However, the embodiments may be implemented in various different forms and are not limited to examples described herein.

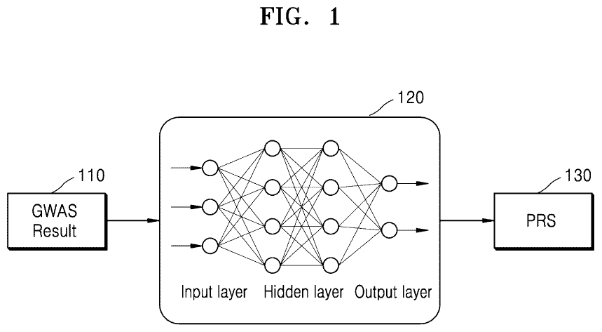

FIG. 1 is a diagram for describing an example of a model for estimating a PRS, according to an embodiment.

Referring to FIG. 1, a device for calculating a PRS (hereinafter, a ‘PRS calculating device’), according to an embodiment of the present disclosure, may calculate a PRS estimation value 130 from a genome-wide association study (GWAS) result 110 by using a model for estimating a PRS (hereinafter, a ‘model’) 120. For example, input data of the model 120 may be the GWAS result 110, and output data of the model 120 may be the PRS estimation value 130.

Here, the GWAS is an exploratory method of finding traits associated with genetic mutations (for example, height, hair color, eye color, risk of various diseases, or the like). In general, the GWAS uses a method of comparing genetic information of a case (a group with a trait of interest, for example, a patient group) with genetic information of a control (a group without a trait, for example, a normal group) across an entire genome region and selecting a genetic mutation with a higher frequency on the case as a genetic mutation associated with the trait.

In other words, when the description of the trait of interest is limited to the occurrence or non-occurrence of a specific disease, the GWAS result 110 may include information about the association between a genetic mutation and the occurrence of a specific disease. That is, the GWAS result 110 may include one or more SNPs which increase the likelihood that an individual will develop a specific disease.

Accordingly, a disease development risk may be predicted based on the GWAS result 110. For example, the disease development risk may be predicted by reflecting mutations in specific genes known to play an important role in the occurrence of diseases, including many genetic mutations identified through the GWAS.

As one example of the method of predicting the disease development risk, there may be a method of calculating a PRS. Furthermore, the PRS may be estimated by using a deep learning model. For example, the model 120 may be a deep learning model including an input layer, an output layer, and a plurality of hidden layers between the input layer and the output layer.

For example, the model 120 may receive an SNP list including one or more SNPs and output a PRS estimation value based on the SNP list. Here, each SNP included in the SNP list may constitute a node of the input layer. Furthermore, each hidden layer may transmit an output to a next layer through a series of linear operations or activation functions by using input data. The output layer may finally output a PRS estimation value by performing the above-described operation.

In this process, the model 120 may assign a weight to each SNP included in the SNP list and output the PRS estimation value 130 based on the assigned weight. For example, the model 120 may receive an SNP list including three SNPs, SNP IDs of which are rs1234, rs5678, and rs9101, respectively. For example, in case that the model 120 has been trained to output a high PRS in response to the inclusion of the rs5678 SNP in the input data during the learning operation, the model 120 may assign a high weight to the rs5678 SNP. As another example, in case that the model 120 has been trained to output a low PRS in response to the inclusion of the rs1234 SNP in the input data during the learning operation, the model 120 may assign a low weight to the rs1234 SNP. As in the example described above, the model 120 may assign a weight to an SNP included in the received SNP list according to the training in the learning operation, and may output the PRS estimation value 130 based on the weight.

As described above, the PRS calculating device according to an embodiment of the present disclosure may calculate the PRS estimation value 130 by using the deep learning model and calculate the PRS by reflecting interaction between the respective SNPs included in the SNP list.

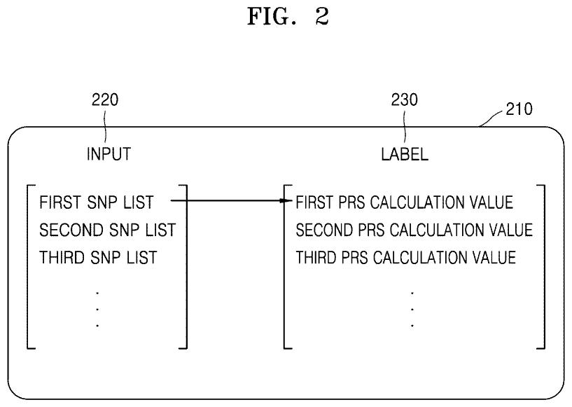

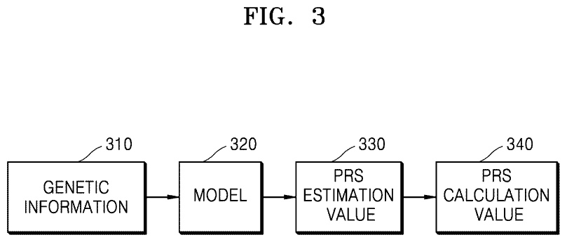

Hereinafter, an example of a method of training a model to output a PRS estimation value by appropriately reflecting interaction between SNPs is described with reference to FIGS. 2 and 3.

FIG. 2 is a diagram for describing an example of a training data set used to train a model, according to an embodiment.

Referring to FIG. 2, a device for training a model for estimating a PRS (hereinafter, a ‘device’) may configure a training data set 210 including one or more pieces of training data in which genetic information about an SNP is an input 220 and a PRS calculation value corresponding to the genetic information is a label 230.

For example, the genetic information about the SNP may be an SNP list including one or more SNPs. More specifically, the device may generate an SNP list from data regarding the GWAS based on a certain P-value, and may use the generated SNP list as the input 220 of the training data set 210. For example, the device may determine the certain P-value for selecting the SNPs and may generate the SNP list by selecting one or more SNPs having data significance in the data regarding the GWAS, based on the determined P-value.

For example, the training data set 210 may include training data in which a first SNP list having a P-value less than 0.0003 is the input 220 and a first PRS calculation value corresponding to the first SNP list is the label 230. As another example, the training data set 210 may include training data in which a second SNP list having a P-value less than 0.0002 is the input 220 and a second PRS calculation value corresponding to the second SNP list is the label 230.

On the other hand, according to an embodiment, the label 230 included in the training data set 210 may be a disease determination result corresponding to the genetic information. For example, the PRS value may be determined according to the SNP list included in the input 220 of the training data set 210, and the disease determination result based on the determined PRS value may be the label corresponding to the input.

According to another embodiment, the label 230 included in the training data set 210 may be a trait value corresponding to the genetic information. For example, the PRS value may be determined according to the SNP list included in the input 220 of the training data set 210, and the trait value calculated based on the determined PRS value may be the label corresponding to the input. Here, the trait value is a value related to a feature of an individual or an organism and may include values which may be expressed numerically, such as a height value or a weight value, hair color, blood type, and disease resistance values.

FIG. 3 is a diagram for describing an example of a method of training a model based on a training data set, according to an embodiment.

Referring to FIG. 3, a device may train a model 320 for estimating a PRS, based on a training data set.

For example, the device may calculate genetic information 310 by using the model 320. Furthermore, the device may obtain, from the model 320, a PRS estimation value 330 corresponding to the genetic information 310. For example, the PRS estimation value 330 may be an estimation value for the PRS output by the model 320, based on the weight assigned to each SNP included in the SNP list, as described with reference to FIG. 1.

Furthermore, the device may update parameters for the model 320 by performing backpropagation on the model 320, based on a loss function regarding an error between the PRS estimation value 330 and a PRS calculation value 340, which is the label for the genetic information 310. In other words, the device may train the model 320 in a direction that reduces the error between the PRS calculation value 340 included in the training data set and the PRS estimation value 330 output from the model 320. That is, as the model 320 repeats training, the model 320 may update its own parameters (for example, weight, bias, or the like) to output accurate PRS estimation values for the input SNP list. On the other hand, the loss function refers to a function that measures the difference between the prediction value of the model for the PRS and the actual value, that is, the PRS estimation value 330 and the PRS calculation value 340, and the type of function, such as mean squared error (MSE) or cross entropy, is not limited.

On the other hand, the model 320 may learn the weight assigned to each SNP included in the SNP list during the learning process. For example, as in the example described with reference to FIG. 1, in case that the model 320 receives an SNP list including three SNPs, SNP IDs of which are rs1234, rs5678, and rs9101, respectively, the model 320 may be trained to assign a weight of 0.4 to rs1234, a weight of 0.2 to rs5678, and a weight of 0.4 to rs9101 through learning.

Here, the model 320 needs to be trained to assign an appropriate weight to each SNP included in the SNP list by taking into account the association between the SNPs. For example, the rs9101 SNP may be assigned a relatively high weight because of the association with the rs1234 SNP, even though the rs9101 SNP should be assigned a low weight because the rs9101 SNP is not significantly associated with the likelihood that an individual will develop a specific disease.

For example, due to the traits of the genetic information, the SNPs located around the same gene are likely to be highly associated with each other because the SNPs are derived from the same ancestor on the same chromosome. For example, linkage disequilibrium (LD) may be a measure of the genetic association between one SNP and another. For example, the LD may be expressed as a value from 0 to 1. As the LD value is closer to 1, two SNPs may have a stronger association. In the example described above, the rs1234 SNP and the rs9101 SNP may have an LD value close to 1 because the SNPs thereof are located close to each other within the gene.

As another example, the model 320 may receive an SNP list including four SNPs, SNP IDs of which are rs1234, rs5678, rs9101, and rs1121, respectively. In this case, to calculate an appropriate PRS corresponding to the SNP list, the influence of the rs1234 and the rs1121 should be evaluated highly, but the model 320 may evaluate the influence of the rs1234 and the rs9101 highly due to the high association between the rs1234 and the rs9101, based on training. In other words, the model 320 may assign a higher weight to the rs9101 SNP, instead of the rs1121 SNP, due to overfitting to training data. This may ultimately reduce the accuracy of the output of the model 320.

On the other hand, as an example of a method of allowing the model 320 to estimate an accurate PRS value, a method using an LD panel which includes information about LD values between SNPs included in the SNP list may be used. For example, the model 320 may construct a prior distribution based on the LD panel and estimate the PRS value by reflecting the association between SNPs through shrinkage of the distribution of the GWAS result. However, there may be a problem in that the use of the LD panel for this purpose unnecessarily requires excessive data.

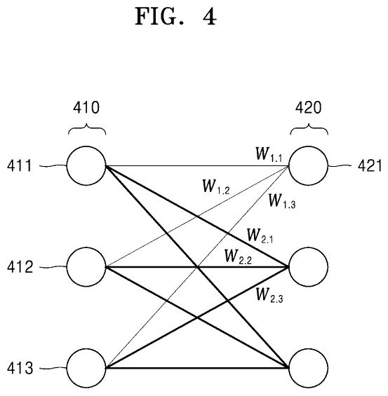

Accordingly, another example of a method of training a model based on a training data set is described below with reference to FIG. 4. More specifically, a model training method described with reference to FIG. 4 relates to a method of training a model to assign a weight to each SNP by taking into account the association between SNPs.

FIG. 4 is a diagram for describing another example of a method of training a model based on a training data set, according to an embodiment.

Referring to FIG. 4, a calculating process between a first layer 410 and a second layer 420, which are layers of a hidden layer constituting the model, is illustrated. For example, a value corresponding to a 2.1 node 421, which is node 1 of the second layer 420, may be calculated based on a plurality of nodes of the first layer 410. Here, w(1,1) may refer to a weight assigned to a value of a 1.1 node 411, which is node 1 of the first layer 410, in calculating the value of the 2.1 node 421 of the second layer 420. Similarly, w(1,2) may refer to a weight assigned to a value of a 1.2 node 412, which is node 2 of the first layer 410, and w(1,3) may refer to a weight assigned to a value of a 1.3 node 413, which is node 3 of the first layer 410. For convenience of explanation, when giving a description excluding a bias value, the value of the 2.1 node 421 may be calculated as the sum of the value of the 1.1 node 411*w(1, 1), the value of the 1.2 node 412*w(1,2), and the value of the 1.3 node 413*w(1,3).

In the example, in case that a higher weight than influence is assigned due to a high association between the 1.1 node 411 and the 1.3 node 413, the device may be adjusted to decrease the weight assigned to the values of the 1.1 node 411 and the 1.3 node 413. In contrast, in case that a lower weight than influence is assigned due to a low association between the 1.1 node 411 and the 1.3 node 413, the device may be adjusted to increase the weight assigned to the values of the 1.1 node 411 and the 1.3 node 410.

To this end, according to an embodiment of the present disclosure, the device may newly define a loss function that guides the training direction of the model. In other words, the device may update parameters for the model (for example, the weight, the bias, or the like assigned to each node) by performing backpropagation on the model based on the gradient of the newly derived loss function.

In an embodiment, the loss function may include a first term which calculates an error between the PRS estimation value and the PRS calculation value and a second term which calculates a correlation between the weights assigned to SNPs input to the model.

For example, the correlation between the weights may be calculated by calculating vector similarity for weight vectors. In other words, a high similarity between the weight vectors may mean a high association between the corresponding SNPs. For example, the second term described above may refer to a term related to the correlation between the weights to be taken into account so as to ensure that an upper bound of a generalization error is a certain level or less, based on the probably approximately correct-Bayesian (PAC-Bayesian) framework.

More specifically, the device may calculate a prior distribution matrix based on the correlation between the weights assigned to the SNPs input to the model. For example, the device may calculate an average similarity between all weight vectors by calculating the similarity between the weight vectors assigned to the SNPs through cosine similarity, and may calculate the prior distribution matrix by using the average similarity.

Furthermore, the device may calculate a posterior distribution matrix based on a covariance of the prior distribution matrix and the weight vector. For example, the device may calculate the posterior distribution matrix through a Kronecker product operation on the prior distribution matrix and a covariance matrix of the weight vectors.

Furthermore, the device may derive a second term which calculates the difference in information amount between the prior distribution matrix and the posterior distribution matrix, based on Kullback-Leibler divergence. In other words, the second term of the loss function may be a term related to the average correlation of the weight vector distribution assigned to each node corresponding to the SNP.

On the other hand, the device may update parameters for the model (for example, the weight, the bias, or the like assigned to each node) by performing backpropagation on the model based on the gradient of the loss function, as described above.

For example, the device may calculate the gradient of the loss function by calculating the gradient for the first term and the second term of the loss function, and may update the parameters for the SNPs input to the model based on the calculated gradient.

In case that the gradient of the loss function is negative, weights for current model parameters may be increased according to gradient descent. In contrast, in case that the gradient of the loss function is positive, weights for current model parameters may be decreased according to gradient descent.

For example, in case that the gradient value calculated by the device for each of the first term and the second term is negative, the device may increase the weight for the SNP input to the model. As another example, in case that the gradient value calculated for each of the first term and the second term is positive, the device may decrease the weight for the SNP input to the model. In the example, the device may update the parameters for the model in a direction that reduces the correlation between the weights assigned to the SNPs by adjusting the weights for the SNPs according to the gradient of the second term.

On the other hand, even when the gradient value calculated for the first term is positive, in case that the gradient value calculated for the second term is negative with a greater absolute value, the gradient calculated for the entire loss function may be negative. Furthermore, even when the gradient value calculated for the first term is negative, in case that the gradient value calculated for the second term is positive with a greater absolute value, the gradient calculated for the entire loss function may be positive. In this case, the device may update the parameters of the model based on the gradient of the entire loss function including the first term and the second term.

For example, in case that the gradient of the entire loss function is positive, the device may decrease the weight for the SNP input to the model. As another example, in case that the gradient of the entire loss function is negative, the device may increase the weight for the SNP input to the model. In other words, according to an embodiment of the present disclosure, the device may adjust weights for current model parameters according to a descent method into which the weight correlation is taken into account.

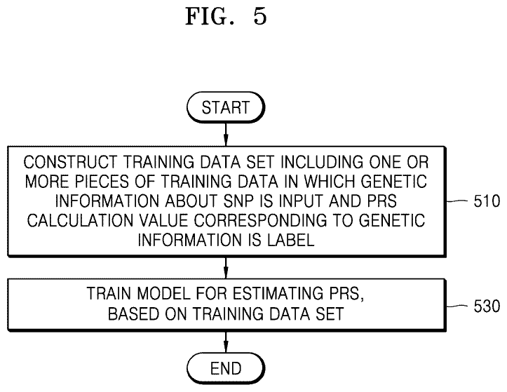

FIG. 5 is a flowchart for describing an example of a method of training a model for estimating a PRS, according to an embodiment.

Referring to FIG. 5, the method of training a model for estimating a PRS may include operations 510 to 530. However, even when omitted below, the description described with reference to FIGS. 1 to 4 may also be applied to the method of training a model for estimating a PRS in FIG. 5.

In operation 510, the device may construct a training data set including one or more pieces of training data in which genetic information about an SNP is an input and a PRS calculation value corresponding to the genetic information is a label.

According to an embodiment, the device may generate an SNP list from data regarding a GWAS based on a certain P-value, and may construct a training data set by using the generated SNP list as an input of the training data set.

In operation 530, the device may train a model for estimating a PRS, based on the training data set.

For example, the device may calculate the genetic information using the model, obtain the PRS estimation value corresponding to the genetic information from the model, and update parameters for the model by performing backpropagation on the model based on a loss function regarding an error between a PRS estimation value and the PRS calculation value, which is the label for the genetic information.

In an embodiment, the loss function may include a first term which calculates an error between the PRS estimation value and the PRS calculation value and a second term which calculates a correlation between the weights assigned to SNPs input to the model.

More specifically, the device may derive the second term which calculates a prior distribution matrix based on the correlation between the weights assigned to the SNPs input to the model, calculates a posterior distribution matrix based on a covariance matrix of the prior distribution matrix and weight vectors, and calculates the difference in information amount between the prior distribution matrix and the posterior distribution matrix based on Kullback-Leibler divergence.

On the other hand, the device may calculate the gradient of the loss function by calculating the gradient for the first term and the second term of the loss function, and may update the parameters for the SNPs input to the model based on the calculated gradient.

For example, in case that the gradient of the loss function is positive, the device may decrease the weight for the SNP input to the model, and in case that the gradient of the loss function is negative, the device may increase the weight for the SNP input to the model.

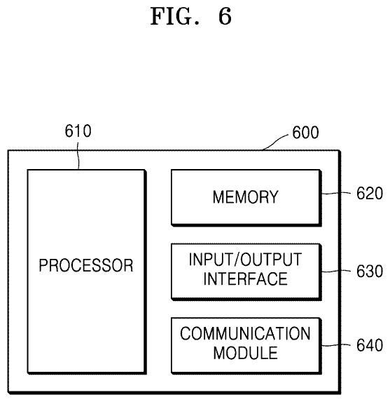

FIG. 6 is a configuration diagram for describing an example of an internal configuration of a device for training a model for estimating a PRS, according to an embodiment.

Referring to FIG. 6, a device 600 may include a processor 610, a memory 620, an input/output interface 630, and a communication module 640. For convenience of explanation, only components related to the present disclosure are illustrated in FIG. 6. Accordingly, in addition to the components illustrated in FIG. 6, other general-purpose components may be further included in the device 600. It is obvious to those of ordinary skill in the art that the processor 610, the memory 620, the input/output interface 630, and the communication module 640 illustrated in FIG. 6 may be implemented as independent devices.

The processor 610 may process commands of a computer program by performing basic arithmetic, logic, and input/output operations. Here, the commands may be provided from the memory 620 or an external device (not shown). Furthermore, the processor 610 may control the overall operations of other components included in the device 600.

The processor 610 may be implemented as an array of a plurality of logic gates, or may be implemented as a combination of a general-purpose microprocessor and a memory storing a program which is executable on the microprocessor. For example, the processor 610 may include a general-purpose processor, a central processing unit (CPU), a microprocessor, a digital signal processor (DSP), a controller, a microcontroller, a state machine, or the like. In some environments, the processor 610 may include an application-specific integrated circuit (ASIC), a programmable logic device (PLD), a field programmable gate array (FPGA), or the like. For example, the processor 610 may refer to a combination of processing devices, such as a combination of a DSP and a microprocessor, a combination of a plurality of microprocessors, a combination of one or more microprocessors connected to a DSP core, or a combination of any other configurations.

The memory 620 may include any non-transitory computer-readable recording medium. As an example, the memory 620 may include a permanent mass storage device, such as random access memory (RAM), read-only memory (ROM), disk drive, solid state drive (SSD), or flash memory. As another example, the permanent mass storage device, such as ROM, SSD, flash memory, or disk drive, may be a separate permanent storage device, which is distinct from memory. Furthermore, the memory 620 may store an operating system (OS) and at least one program code (for example, code for causing the processor 610 to perform operations described with reference to FIGS. 1 to 5).

Such software components may be loaded from a computer-readable recording medium, which is separate from the memory 620. The separate computer-readable recording medium may be a recording medium which may be directly connected to the device 600. For example, the separate computer-readable recording medium may include floppy drive, disk, tape, digital versatile disc (DVD)/compact disc read-only memory (CD-ROM) drive, memory card, or the like. Alternatively, the software components may be loaded into the memory 620 through the communication module 640 rather than the computer-readable recording medium. For example, the at least one program may be loaded into the memory 620, based on a computer program installed by files provided through the communication module 640 by developers or a file distribution system which distributes an application installation file (for example, a computer program for causing the processor 610 to perform operations described with reference to FIGS. 1 to 5).

The input/output interface 630 may be a means for interfacing with an input or output device (for example, a keyboard, a mouse, or the like) which may be connected to or included in the device 600. FIG. 6 illustrates that the input/output interface 630 is an element configured separately from the processor 610, but the present disclosure is not limited thereto, and the input/output interface 630 may be configured to be included in the processor 610.

The communication module 640 may provide a configuration or a function for the server and the device 600 to communicate with each other via a network. Furthermore, the communication module 640 may provide a configuration or a function for the device 600 to communicate with other external devices. For example, control signals, commands, data, and the like, which are provided under the control of the processor 610, may be transmitted to the server and/or an external device via the communication module 640 and the network.

Embodiments according to the present disclosure may be implemented in the form of a computer program that may be executed through various elements on a computer, and such a computer program may be recorded on a computer-readable medium. At this time, the medium may include a magnetic medium such as hard disk, floppy disk, and magnetic tape, an optical recording medium such as CD-ROM and DVD, a magneto-optical medium such as floptical disks, and hardware devices specially configured to store and execute program instructions, such as ROM, RAM, or flash memory.

The computer program may be specially designed and configured for the present disclosure or may be known and available to those of ordinary skill in the art of computer software. Examples of the computer program may include not only machine language code generated by a compiler but also high-level language code that is executable using an interpreter or the like by a computer.

According to an embodiment, the methods according to various embodiments of the present disclosure may be provided by being included in a computer program product. The computer program product may be traded between a seller and a buyer as commodities. The computer program product may be distributed in the form of a machine-readable storage medium (for example, CD-ROM), or may be distributed (for example, downloaded or uploaded) online, either via an application store (for example, Play Store™) or directly between two user devices. In the case of the online distribution, at least part of the computer program product may be stored at least temporarily on a machine-readable storage medium, such as a server of a manufacturer, a server of an application store, or a memory of a relay server, or may be temporarily generated.

Operations constituting methods according to the present disclosure may be performed in any suitable order unless otherwise indicated herein or otherwise clearly contradicted by context. The present disclosure is not necessarily limited by the order of operations. The use of any and all examples or exemplary terms (for example, “such as”) provided herein is simply intended to describe the present disclosure in detail, and the scope of the present disclosure is not limited by the examples or exemplary terms unless otherwise claimed. In addition, it will be understood by those of ordinary skill in the art that various modifications, combinations and changes may be made according to design conditions and factors within the scope of the appended claims or equivalents thereof.

Therefore, it will be understood that the spirit of the present disclosure should not be limited to the embodiments described above, and the claims and all equivalent modifications fall within the scope of the present disclosure.

1. A method of training a model for estimating a polygenic risk score (PRS), the method comprising:
constructing a training data set comprising one or more pieces of training data in which genetic information about a single nucleotide polymorphism(SNP) is an input and a PRS calculation value corresponding to the genetic information is a label; and
training the model for estimating the PRS, based on the training data set,
wherein the training comprises:
calculating the genetic information by using the model;
obtaining, from the model, a PRS estimation value corresponding to the genetic information; and
updating parameters for the model by performing backpropagation on the model, based on a loss function regarding an error between the PRS estimation value and the PRS calculation value, which is the label for the genetic information.

2. The method of claim 1, wherein the constructing of the training data set comprises generating an SNP list based on a certain P-value from data regarding genome-wide association study (GWAS), the generated SNP list being used as the input of the training data.

3. The method of claim 1, wherein the loss function comprises a first term which calculates an error between the PRS estimation value and the PRS calculation value and a second term which calculates a correlation between weights assigned to SNPs input to the model.

4. The method of claim 3, wherein the training comprises:
calculating a prior distribution matrix based on the correlation between the weights assigned to the SNPs input to the model;
calculating a posterior distribution matrix based on a covariance matrix of the prior distribution matrix and a weight vector; and
deriving the second term which calculates a difference in information amount between the prior distribution matrix and the posterior distribution matrix, based on Kullback-Leibler divergence.

5. The method of claim 3, wherein the updating of the parameters comprises:
calculating a gradient of the loss function by calculating gradients for the first term and the second term of the loss function; and
updating the parameters based on the calculated gradient.

6. The method of claim 5, wherein the updating of the parameters comprises decreasing the weight for the SNP input to the model in case that the gradient of the loss function is positive, and increasing the weight for the SNP input to the model in case that the gradient of the loss function is negative.

7. A device for training a model for estimating a polygenic risk score (PRS), the device comprising:
a memory in which at least one program is stored; and
a processor configured to execute the at least one program,
wherein the processor is further configured to:
construct a training data set comprising one or more pieces of training data in which genetic information about a single nucleotide polymorphism(SNP) is an input and a PRS calculation value corresponding to the genetic information is a label; and
train the model for estimating the PRS, based on the training data set,
wherein the processor is further configured to:
calculate the genetic information by using the model;
obtain, from the model, a PRS estimation value corresponding to the genetic information; and
update parameters for the model by performing backpropagation on the model, based on a loss function regarding an error between the PRS estimation value and the PRS calculation value, which is the label for the genetic information.

8. A computer-readable recording medium having recorded thereon a program for causing a computer to perform the method of claim 1.
